(19) United States
(12) Patent Application Publication
(10) Pub. No.: US 2007/0161043 A1
(43) Pub. Date: Jul. 12, 2007

(54) Methods of Preparing Multicolor Quantum Dot Tagged Beads and Conjugates Thereof

(75) Inventors: Shuming Nie, Atlanta, GA (US); Xiaohu Gao, Decatur, GA (US); Mingyong Han, Commonwealth Close (SG)

Correspondence Address: LEYDIG VOIT & MAYER, LTD, TWO PRUDENTIAL PLAZA, SUITE 4900, 180 NORTH STETSON AVENUE, CHICAGO, IL, 60601-6731, US

(73) Assignee: Advanced Research and Technology Institute, Inc., Indianapolis, IN (US)

(21) Appl. No.: 11/566,601

(22) Filed: Dec. 4, 2006

Related U.S. Application Data
(63) Continuation of application No. 10/185,226, filed on Jun. 28, 2002, now abandoned.
(60) Provisional application No. 60/301,573, filed on Jun. 28, 2001.

(51) Int. Cl. G01N 33/53; B05D 3/00

(52) U.S. Cl. 435/7.1; 427/2.11; 977/902

(57) ABSTRACT

The present invention provides a method of preparing a multicolor quantum dot-tagged bead, a multicolor quantum dot-tagged bead, a conjugate thereof, and a composition comprising such a bead or conjugate. Additionally, the present invention provides a method of making a conjugate thereof and methods of using a conjugate for multiplexed analysis of target molecules.

### CROSS-REFERENCE TO RELATED PATENT APPLICATIONS

This patent application is a continuation of U.S. patent application Ser. No. 10/185,226, filed on Jun. 28, 2002, which claims the benefit of U.S. Provisional Patent Application No. 60/301,573, filed Jun. 28, 2001, all of which are hereby incorporated in its entirety by reference.

### GOVERNMENT SUPPORT

This invention was made in part with Government support under Grant Numbers R01GM60562 and FG02-98ER14873 awarded by the National Institutes of Health and the Department of Energy. The Government may have certain rights in this invention.

### TECHNICAL FIELD OF THE INVENTION

The present invention relates to methods of obtaining a multicolor quantum dot-tagged bead, multicolor quantum dot-tagged beads, a conjugate thereof and a composition comprising such a quantum dot-tagged bead or conjugate. Additionally, the present invention relates to methods of using a conjugate for multiplexed detection of targets, in particular biomolecular targets.

### BACKGROUND OF THE INVENTION

Recent advances in bioanalytical sciences and bioengineering have led to the development of DNA chips, miniaturized biosensors and microfluidic devices. In addition, applications benefiting from fluorescent labeling include medical (and non-medical) fluorescence microscopy, histology, flow cytometry, fundamental cellular and molecular biology protocols, fluorescence in situ hybridization, DNA sequencing, immuno assays, binding assays and separation. These enabling technologies have substantially impacted many areas in biomedical research, such as gene expression profiling, drug discovery, and clinical diagnostics.

Fluorescently-labeled molecules have been used extensively for a wide range of applications. Typically organic dyes are bonded to a probe, which in turn selectively binds to a target. The target is then identified by exciting the dye molecule, causing it to fluoresce. There are many disadvantages to using an organic dye for these fluorescent-labeling systems. The emission of visible light from an excited dye molecule usually is characterized by the presence of a broad emission spectrum (about 100 nm) and broad tails of emission at red wavelengths (about another 100 μm). As a result, there is a severe limitation on the number of different color organic dye molecules which can be utilized simultaneously or sequentially in an analysis since it is difficult to either simultaneously or even non-simultaneously detect or discriminate between the presence of a number of different detectable substances due to the broad spectrum emissions and emission tails of the labeling molecules. Another problem is that organic dyes often have a narrow absorption spectrum (about 30-50 nm), thus requiring either multiple wavelength probes, or else broad spectrum excitation source which is sequentially used with different filters for sequential excitation of a series of probes respectively excited at different wavelengths.

Another problem associated with organic dyes is their lack of photostability. Often organic dyes bleach or cease to fluoresce under repeated excitation. These problems are often overcome by minimizing the amount of time that the sample is exposed to the light source and by removing any radical species (including oxygen) from the sample.

It would be desirable to provide an assay of identifying target molecules, which takes advantage of tags that emit visible light, have narrow emissions, broad excitations, and are photostable. Using luminescent semiconductor quantum dots as fluorescent tags has been a useful approach in identifying targets, such as biomolecules. In comparison to an organic dye (e.g., Rhodamine), quantum dots are 20 times as bright, approximately 100 times as photostable, and have emission spectra that are approximately one third the width. These desirable properties allow for the simultaneous use of quantum dots of different emission wavelengths (i.e., colors) while preserving the ability to resolve them from each other. In addition, the broad excitation spectrum allows many different quantum dots to be excited by a common light source.

Over the past decade, much progress has been made in the synthesis and characterization of a wide variety of semiconductor quantum dots. Recent advances have led to large-scale preparation of relatively monodisperse quantum dots (Murray et al., J. Am. Chem. Soc., 115, 8706-15 (1993); Bowen Katari et al., J. Phys. Chem., 98, 4109-17 (1994); and Hines et al., J. Phys. Chem., 100, 468-71 (1996)). Other advances have led to the characterization of quantum dot lattice structures (Henglein, Chem. Rev., 89, 1861-73 (1989); and Weller et al., Chem. Int. Ed. Engl. 32, 41-53(1993)) and also to the fabrication of quantum-dot arrays (Murray et al., Science, 270, 1335-38 (1995); Andres et al., Science, 273, 1690-93 (1996); Heath et al., J. Phys. Chem., 100, 3144-49 (1996); Collier et al., Science, 277, 1978-81 (1997); Mirkin et al., Nature, 382, 607-09 (1996); and Alivisatos et al., Nature, 382, 609-11 (1996)) and light-emitting diodes (Colvin et al., Nature, 370, 354-57 (1994); and Dabbousi et al., Appl. Phys. Let., 66, 1316-18 (1995)). In particular, IIB-VIB semiconductors have been the focus of much attention, leading to the development of a CdSe quantum dot that has an unprecedented degree of monodispersity and crystalline order (Murray (1993), supra).

The potential of multiplexed coding (e.g., using multiple wavelengths and multiple intensities) has also been recognized by other researchers (see, e.g., WO 99/37814, WO 01/13119, WO 01/13120, WO 99/19515, WO 97/14028). For example, Fulton et al. used two organic dyes to encode a set of about 100 beads for multiplexed and multianalyte bioassays (see Fulton, R. J., et al., Clin. Chem. 43, 1749-1756 (1997)). Walt and coworkers reported randomly ordered fiber-optic microarrays based on fluorescently encoded microspheres (see Steemers, R. J., et al. Nature Biotechnol. 18, 91-94 (2000); Ferguson, J. A., et al. Nature Biotechnol. 14, 1681-1684 (1996); Ferguson, J. A., et al. Anal. Chem. 72, 5618-5624 (2000)). However, these previous studies were based on organic dyes and lanthanide complexes, and were limited by the unfavorable absorption and emission properties of these materials (e.g., inability to excite more than 2-3 fluorophores, broad and asymmetric emission profiles, and spectral overlapping).

Systems comprising two (or more) organic dyes embedded in beads are prone to fluorescence resonance energy transfer (FRET), the emission spectra of the beads with the organic dyes embedded are not predictable and therefore prove unreliable, and cannot be detected by a wavelength-resolved spectroscopy combined with a microchannel. Moreover, organic dyes cannot have continuously tunable emission wavelengths. Finally, because different organic dyes are soluble in solvents to varying degrees of solubility, the dyes cannot be embedded in the beads in a precisely controlled ratio. The ratio of dyes cannot be predetermined before incorporation. This drawback severely limits the number of beads useful for multiplexed analysis of targets.

Recent approaches for associating quantum dots with substrates, such as beads, in order to detect biomolecular targets have been disclosed (see, for example, WO 01/71044, WO 00/71995, WO 01/13119, and WO 99/47570). However, none of these approaches provide beads that contain quantum dots embedded therein in a precisely controlled ratio and reproducible manner. For example, WO 01/71044 discloses attaching dihydrolipoic acid-capped, water-soluble quantum dots to commercially available polymeric beads in an aqueous solution. Because there are more carboxylic groups on the bead's surface compared to its interior, the hydrophilic quantum dots would prefer to stay ill the aqueous solution surrounding the bead's exterior. Furthermore, since the number and size (i.e., color) of quantum dots that enter the bead's interior versus those that remain on the bead surface cannot be controlled, the resulting quantum dot-tagged beads are not very reproducible compared to each other and batch to batch. Typically, the number of QDs associated with the bead is quite low. In addition, WO 01/71044 discloses heating the polymer beads and quantum dots in a large amount of chloroform in order to swell the beads. Exposing water-soluble quantum dots to heat causes the QDs to become unstable.

As current research in genomics and proteomics produces more sequence data, there is a strong need for new and improved technologies that can rapidly screen a large number of nucleic acids and proteins. From the foregoing it will be appreciated that while organic dyes have been useful in the past for the detection of biomolecules, there is a need for more accurate, more sensitive, and broader methods of detection, which includes a method of multiplexed analysis of multiple targets.

### BRIEF SUMMARY OF THE INVENTION

Towards the ultimate goal of better molecular target detection, the present invention permits an optical coding technology, preferably multiplexed optical coding. Such a technology allows for “lab-on-a-bead” for massively parallel and high throughput analysis of targets, in particular biological molecules. This technology is premised, at least in part, on the novel optical properties of semiconductor quantum dots and the ability to incorporate multicolor quantum dots into beads at precisely controlled ratios. Based on the ratio of quantum dots added, a unique identifiable code exists for each bead. The multicolor quantum dot-tagged beads can then be converted into a conjugate by attaching a probe to the bead. This conjugate can combine with a target, allowing for facile identification of the target.

Thus, in one aspect, the present invention provides a quantum dot-tagged bead comprising at least one quantum dot and a porous bead. The bead has pores large enough to permit entry of the quantum dot therethrough and into the bead. Preferably, the quantum dots are present in a predetermined precisely controlled ratio.

The present invention also provides methods of preparing a multicolor quantum dot-tagged bead. Also provided is a multicolor quantum dot-tagged bead prepared by the methods and compositions comprising the multicolor luminescent quantum dot-tagged bead and a carrier. The present invention further provides a conjugate, which comprises the multicolor quantum dot-tagged bead prepared by the method and a probe, wherein the probe is attached directly or indirectly to the bead. Also provided is a composition comprising the conjugate and a carrier. Further provided by the present invention are methods of making conjugates thereof and methods of detecting targets with multicolor quantum dot-tagged beads.

Compared to coding systems that use organic dyes, the present invention has a number of advantages: the fluorescence emission wavelength can be continuously tuned, a single wavelength can be used for simultaneous excitation of all different colored quantum dots, the emission spectra are narrow allowing for multiple colors (i.e., wavelengths) to be used, there is no fluorescence resonance energy transfer (FRET) between the quantum dots, and the quantum dots are photostable.

The present invention also has advantages over organic dye systems in that it allows for multiplexed analysis of a large number of targets. The analysis is aided by the high stability of multicolor quantum dot-tagged beads and their ease of preparation, modification, and detection. In comparison with planar DNA chips, the encoded bead technology of the present invention is expected to be more flexible in target selection, faster in binding kinetics (similar to that in homogeneous solution), and cheaper in production. These and other objects and advantages, as well as additional inventive features, of the present invention will become apparent to one of ordinary skill in the art upon reading the detailed description provided herein.

### BRIEF DESCRIPTION OF THE FIGURES

FIG. 1 is a schematic illustration of optical coding based on wavelength and intensity multiplexing. Large spheres represent polymer microbeads, in which small colored spheres (multicolor quantum dots) are embedded according to pre-determined intensity ratios. “\” Cross-hatchings indicate red quantum dots, “/” cross-hatchings indicate green quantum dots, and “X” cross-hatchings indicate blue quantum dots. Molecular probes (A to E) are attached to the bead surface for biological binding and recognition, such as DNA-DNA hybridization and antibody-antigen/ligand-receptor interactions. The numbers of colored spheres (red, green, and blue) do not represent individual quantum dots, but are used to illustrate the fluorescence intensity levels. Optical readout is accomplished by measuring the fluorescence spectra of single beads. Both absolute intensities and relative intensity ratios at different wavelengths are used for coding purposes; for example, (1:1:1), (2:2:2) and (2:1:1) are distinguishable codes.

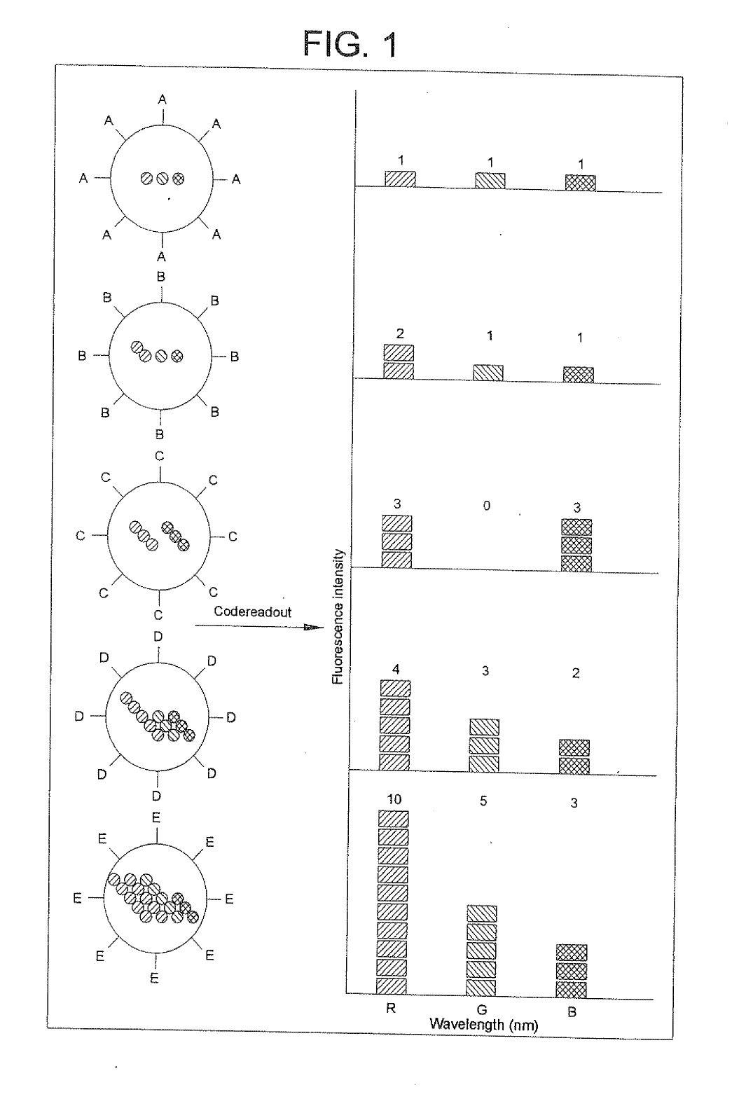

FIG. 2 is the quantitative analysis of single-bead signal intensities, uniformity and reproducibility of QD incorporation. (A) Relationship between the fluorescence intensity of a single bead and the number of embedded QDs. Each data point is the average value of 100 to 200 measurements, and the signal intensity spread (minimum-to-maximum) is indicated by an error bar. The first point (lowest intensity) corresponds to about 640 dots per bead. The last point shows a significant deviation from the linear line because of incomplete incorporation of QDs into the beads at this loading level. (B) Histogram plots for 10 intensity levels corresponding to the data points in (A). On the right side of each curve is shown the average fluorescence intensity as well as the standard deviation (in parenthesis). Representative raw data are shown for levels 2 and 8.

FIG. 3 is a schematic representation of a working curve prepared for more than one color. The dotted line represents how a bead with a 1:1:1 code would be formulated. The solvent concentrations of blue (“B”), green (“G”), and red (“R”) quantum dots can be determined from the X axis.

FIG. 4 depicts multicolor QD-tagged beads with precisely controlled fluorescence intensities. (A) Fluorescence image of color-balanced beads. In the upper right corner, single-color beads were digitally inserted to show that this should not be mistaken as a black and white image. “\” Cross-hatchings indicate red quantum dots, “\” cross-hatchings indicate green quantum dots, and “X” cross-hatchings indicate blue quantum dots. (B) Single-bead fluorescence spectrum, showing three separated peaks (484, 547, and 608 mm) with nearly equal intensities. “B” stands for blue; “G” stands for green, and “R” stands for red.

FIG. 5 is a schematic illustration of DNA hybridization assays using QD-tagged beads. Probe oligos (No. 1-4) were conjugated to the beads by cross-linking, and target oligos (No. 1-4) were detected with a blue fluorescent dye such as Cascade Blue (labeled “F”). “\” Cross-hatchings indicate red quantum dots, “/” cross-hatchings indicate green quantum dots, and “X” cross-hatchings indicate blue quantum dots. After hybridization, nonspecific molecules and excess reagents were removed by washing. For multiplexed assays, the oligo lengths and sequences were optimized so that all probes had similar melting temperatures and hybridization kinetics.

FIG. 6 depicts DNA hybridization assays using multicolor encoded beads. (A) Fluorescence signals obtained from a single bead with the code 1:1:1 (corresponding to probe 5′-TCA AGG CTC AGT TCG AAT GCA CCA TA-3′), after exposure to a control DNA sequence (3′-TGA TTC TCA ACT GTC CCT GGA ACA GA-5′). The control DNA was tagged with the same fluorophore as the target DNA. (B) Fluorescence signals of a single bead with the code 1:1:1 [same as in (A)], after hybridization with its target 5′-TAT GGT GCA TTC GAA crG AGC CTT GA-3′. (C) Fluorescence signals of a single bead with the code 1:2:1 (corresponding to probe 5′-CCG TAC AAG CAT GGA ACG GCT TTT AC-3′), after hybridization with its target 5′-GTA AAA GCC GTT CCA TGC TTG TAC GG-3′. (D) Fluorescence signals of a single bead with the code 2:1:1 (corresponding to probe 5′-TAC TCA GTA GOCG ACA CAT OGT TCG AC-3′), after hybridization with its target 5′-GTC GAA CCA TGT GTC GCT ACT GAG TA-3′.

FIG. 7 depicts a schematic illustration of a molecular beacon. “\” Cross-hatchings indicate red quantum dots, “/” cross-hatchings indicate green quantum dots, and “X” cross-hatchings indicate blue quantum dots. The multicolor quantum dot-tagged bead can be attached to either the fluorophore (A) or the quenching moiety (B).

### DETAILED DESCRIPTION OF THE INVENTION

The present invention provides a multicolor quantum dot-tagged bead, conjugates thereof, and methods, diagnostic libraries, and molecular beacons related thereto. In accordance with preferred embodiments of the invention, various probes can be directly and indirectly attached to a multicolor quantum dottagged bead to provide massively parallel and high-throughput analysis of molecules, particularly biological molecules.

In one aspect, the present invention provides a method of preparing a multicolor quantum dot-tagged bead. In general, a method of preparing a multicolor quantum dot-tagged bead comprises the steps of (a) providing at least one porous bead, wherein the pores of the bead are large enough to incorporate quantum dots; (b) combining predetermined amounts of multicolor quantum dots with at least one bead; and (c) isolating the multicolor quantum dot-tagged bead.

In another aspect, the present invention provides a multicolor quantum dot-tagged bead, which comprises at least one multicolor quantum dot and a porous polymer bead, wherein the bead has pores large enough to incorporate the quantum dot, and wherein the quantum dots are present in a precisely controlled ratio. By the term “porous” it is meant that the bead has openings on the surface and within its interior that are large enough for a quantum dot to pass through and into the interior of the bead. For clarity of description, beads that are sealed with a sealant compound after the multicolor quantum dots are embedded through pores are still considered porous for purposes of the present invention.

The bead having pores large enough to incorporate quantum dots can be provided in any suitable manner. For example, in some embodiments, the porous polymer bead is synthesized by emulsion polymerization, suspension polymerization, or seeded polymerization. The ordinary skilled artisan will understand that a particular method described herein can be especially suited for a particular embodiment, and each method for generating the bead has unique advantages. In general, it is desirable to synthesize the polymer beads using methods set forth herein, some of which are based on procedures within the skill of the ordinarily skilled artisan (see, e.g., Ferguson, J. A., et al., Anal. Chem. 72, 5618-5624 (2000)). Emulsion polymerization can occur by any method, such as methods known in the art. For example, a standard method utilizes an oil and water emulsion to polymerize monomer (and any cross-linkers) in the presence of an initiator. Suspension polymerization can occur by any suitable method. One example includes dissolving a stabilizer in an ethanol/water solution. Initiator is dissolved in the monomer, and the monomer-initiator mixture is combined with the ethanol/water solution. The seeded polymerization can occur by as many steps as needed, for example one or two steps. In general, however, small polymer beads are grown to larger diameters in the presence of monomer, initiator, and emulsifier.

Beads according to the invention are sufficiently porous to permit passage of quantum dots into the internal structure of the bead, as quantum dots are relatively larger than organic dye molecules. Preferably, the beads are macroporous. By “macroporous”, it is meant that the pores of the bead have an average diameter of at least about 1 nm. More preferably, the pores have an average diameter of from about 1 nm to about 20 nm, more preferably from about 2 nm to about 10 nm. In some embodiments, the pores have an average diameter of from about 2 μm to about 5 nm. Typically, conventional, commercially available beads do not allow for embedding the QDs, probably due to a lack of porosity or ability to swell appreciably in solution, both of which are likely due to high amounts of cross-linking. Because conventional commercially available beads are not porous, those in the art often use a high concentration of chloroform (e.g., 40-50%) in an attempt to swell the bead. The excessive amount (e.g., 40% v/v) of chloroform typically can damage the bead. Porous beads, according to the invention, can be swollen, but require significantly lower amounts (e.g., less than about 10% v/v, preferably about 5% v/v) of a swelling agent (e.g., chloroform, butanol). Moreover, commercially available beads typically do not have a hydrophobic interior, thereby further inhibiting the incorporation of QDs, particularly hydrophobic QDs.

The porous beads typically are washed with a solution, preferably an alcohol such as ethanol, propanol, and butanol, several times to dehydrate the beads before QD incorporation in solution (preferably also an alcohol solution). The QDs can be incorporated into the beads in any suitable manner. By way of example, and not by way of limitation, QDs can be directly incorporated by several different methods: (i) QDs are directly incorporated into macroporous beads, which are generally prepared by seeded emulsion polymerization or suspension polymerization using a monomer, such as a long chain derivative of acrylic acid (e.g., mono-2-methacryloyloxyethyl succinate); (ii) by soaking or ultrasonicating at room temperature or at elevated temperature (preferably room temperature); and (iii) by swelling beads using solvents, followed by QD incorporation.

The solvent for method (iii) is not particularly limited so long as it permits the beads to swell sufficiently to allow for incorporation of various sizes of QDs. Typically, the solvent is organic, such as acyl, aliphatic, cycloaliphatic, aromatic or heterocyclic hydrocarbons or alcohols with or without halogens, oxygen, sulfur, and nitrogen, although in some instances, water or aqueous solutions can be desirable. Examples of useful solvents include, but are not limited to, benzene, toluene, xylene, cyclohexane, pentane, hexane, ligroin, methyl isobutyl ketone, methylacetate, ethylacetate, butylacetate, methyl CELLOSOLVE® (Union Carbide), ethyl CELLOSOLVE® (Union Carbide), butyl CELLOSOLVE® (Union Carbide), diethylene glycol monobutyl ether, diethylene glycol monobutyl ether acetate, alcohol (e.g., methanol, ethanol, n-propanol, i-propanol, n-butanol, t-butanol, n-pentanol, n-hexanol, branched hexanol, cyclohexanol, 2-ethylhexyl alcohol), acetone, DMSO, methylene chloride, chloroform, and combinations thereof. Preferably, the solvent is alcohol, and more preferably it is a C₃-C₆ linear or branched alcohol. In a most preferred embodiment, the solvent is butanol (normal or tertiary), and the bead is a cross-linked polymer derived from styrene/divinylbenzene/acrylic acid.

Monodispersed QDs with fluorescence emissions of various colors (e.g., red, green, blue) are incorporated into the bead structure according to any of the above-described methods. Typically, the QDs are embedded either sequentially or in parallel. For these procedures to be successful, the distribution of pore sizes within the beads desirably is carefully controlled. The ratio of QDs embedded in the beads arises from careful addition of predetermined amounts of each color.

Preferably, the QDs are sequentially incorporated into the beads. For example, the QDs are embedded one color at a time. The order of addition is not limited. For example, the largest diameter (e.g., red) are added first, the next largest (e.g., green) are added and so on until the smallest (e.g., blue) are added. Alternatively, the QDs are added starting with the smallest diameter, sequentially adding the next largest QDs, and ending with the largest diameter QDs. In some embodiments, the method of incorporating multicolor QDs in beads comprises (a) optionally swelling the beads in a solvent if the pores are not large enough; (b) adding a predetermined amount of QDs of a desired color to the solvent; (c) repeating (b) until all the desired amount of QDs of the desired colors are embedded; and (d) isolating the multicolor quantum dot-tagged bead.

Alternatively, the method includes (b) soaking the beads in one solution comprising each desired color of QD in the desired ratio. The beads are soaked in the solution such that complete parallel incorporation of the multicolor QDs occurs, after which the multicolor quantum dot-tagged bead is isolated.

Rather than soaking the beads in solution to incorporate the QDs, the beads can be ultrasonicated in a solution containing the QDs. Again, incorporation of QDs by ultrasonication can be done sequentially or in parallel.

The number of QDs per bead preferably ranges from 1 to about 60,000. More preferably, the number of QDs per bead is from about 100-50,000, and most preferably from about 600 to about 40,000. The number of QDs per bead is calculated by dividing the total number of QDs by the total number of beads in the mixture, under the assumption that the incorporation process is complete (i.e., there are no free QDs in the supernatant). Fluorescence measurement has confirmed that the incorporation process is complete for low to medium loadings of up to 40,000 QDs per bead. The embedded QDs have similar optical properties as free QDs, and the ratio of these two intensities is approximately equal to the number of QDs per bead. These two independent measurements yield nearly identical results, thereby establishing a linear relationship between the measured fluorescence intensity and the number of embedded QDs.

The bead can be formed from any material(s) but, preferably, the material is stable in a suitable solvent. The bead material can be organic, inorganic, or mixtures thereof. Likewise, the bead can be solid (porous or non-porous) or hollow. Preferably, the bead comprises a solid porous material. It is desirable that the distribution of the pores be carefully controlled. While the beads can be hydrophilic or hydrophobic, the beads of the present invention are preferably hydrophobic. Desirably, if the interior of the bead is hydrophobic, then the QDs incorporated into the interior of the bead are also hydrophobic, and if the interior of the bead is hydrophilic, then the QDs incorporated into the interior of the bead are hydrophilic as well (see, e.g., U.S. patent application Ser. No. 09/405,653, which is incorporated herein by way of reference). The beads can comprise polymer, titanium dioxide, latex or other cross-linked dextrans, cellulose, nylon, cross-linked micelles, Teflon, thoria sol, carbon graphited, resin, ceramic, zeolite, metal and glass. Preferably, the beads are a polymeric material, such as an organic polymer.

Examples of polymeric materials useful for the beads include, but are not limited to, polystyrene, brominated polystyrene, polyacrylic acid, polyacrylonitrile, polyamide, polyacrylamide, polyacrolein, polybutadienoe, polycaprolactone, polycarbonate, polyester, polyethylene, polyethylene terephthalate, polydimethylsiloxane, polyisoprene, polyurethane, polyvinyl acetate, polyvinyl chloride, polyvinyl pyridine, polyvinylbenzyl chloride, polyvinyl toluene, polyvinylidene chloride, polydivinylbenzene, polymethylmethacrylate, polylactide, polyglycolide, poly(lactide-co-glycolide), polyanhydride, polyorthoester, polyphosphazene, polysulfone, and combinations or copolymers thereof. Examples of resins include, for example, hardened rosin, ester gum and other rosin esters, maleic acid resin, fumaric acid resin, dimer rosin, polymer rosin, rosin-modified phenol resin, phenolic resin, xylenic resin, urea resin, melamine resin, ketone resin, coumarone-indene resin, petroleum resin, terpene resin, alkyl resin, polyamide resin, acrylic resin, polyvinyl chloride, vinyl chloride-vinyl acetate copolymer, polyvinyl acetate, ethylene-maleic anhydride copolymer, styrene-maleic anhydride copolymer, methyl vinyl ether-maleic anhydride copolymer, isobutylene-maleic anhydride copolymer, polyvinyl alcohol, modified polyvinyl alcohol, polyvinyl butryl (butryl resin), polyvinyl pyrrolidine, chlorinated polypropylene, styrene resin, epoxy resin, and polyurethane.

The polymer beads can be cross-linked, if desired, with any suitable cross-linking agent known in the art (e.g., divinylbenzene, ethylene glycol dimethacrylate, ethylene glycol diacrylate, trimethylolpropane trimethacrylate, or N,N′ methylene-bis-acrylamide). Generally, about 0.3-30% by volume, preferably about 0.3-5% by volume, and most preferably about 1% by volume of the cross-linking agent (bearing in mind that commercial cross-linking agents are generally about 50-80% active cross-linker) and 20-50% styrene or other monomer are used. A preferred polymeric material for bead construction is polystyrene. Desirably, the polystyrene is cross-linked with divinylbenzene and acrylic acid. The beads preferably have a diameter ranging from about 0.01 μm to about 10 mm. More preferably, the diameter is from about 0.1 μm to about 100 μm, more preferably from about 0.1 μm to about 25 μm, more preferably from about 0.1 μm to about 10 μm, more preferably from about 0.1 μm to about 5 μm, and most preferably from about 0.5 μm to about 5 μm.

As will be described further in the Examples, infra, a solvent system phase can be formulated by mixing about 0.14 g AIBN, about 10 ml styrene, about 100 μl acrylic acid, about 100 μl divinylbenzene, about 10 ml deionized water, about 90 ml ethanol, and about 1 g PVP (polyvinylpyrrolidone, MW=40,000), with degassing and washing. Instead of acrylic acid, included to functionalize the synthesized bead, other polymerizable moieties can be used, depending on the type of functionality desired. For example, monomers that have a terminal COOH, NH₂, OH, or SH functionality can be employed. The approach described in this paragraph is typical of the most preferred method, namely, suspension (also known as precipitation) polymerizations which is a subset of solvent-system polymerization, with a low degree of cross-linking. Solvent-system polymerization is a polymerization in which either a surfactant or any other emulsifying agent is substantially or completely absent, not counting the possible presence of minor amounts of stabilizers. In theory, although there is no intention of being bound by the theory, solvent-system polymerization with a low degree of cross-linking, and more particularly precipitation polymerization with a low degree of cross-linking, first forms discrete polystyrene oligomers, which in turn form limited-chain-length discrete polymer chains having a low number of cross-links between them and, hence, a highly developed labyrinth of pores are created throughout each bead thus formed. The pores of the beads thus created generally have an average diameter of at least about 1 μm, as described elsewhere herein. Beads created by solvent-system polymerization, particularly by precipitation polymerization, are surprisingly well suited to swelling in solvents comprising predominantly linear or branched C₃-C₅ alcohols, such as propanol and/or butanol and/or pentanol. In addition, relatively smaller amounts of a solvent in which polystyrene has a higher solubility, such as, for example, halogenated alkanes (e.g., CH₂Cl₂, CH₃CH₂Cl, CH₃CHCl₂, CH₂Cl—CH₂Cl, CHCl₃), benzene, toluene, dimethyl benzene, ethyl benzene, chlorobenzene, and cholorotoluene, can be added to the swelling solvent. Typical admixtures of this type could include 5% chloroform and 95% of one or more C₃-C₅ alcohol. Shrinking of the beads after swelling may be accomplished as described elsewhere in this specification.

As will be appreciated by the ordinary skilled artisan, the term “quantum dot” (“QD”) in the present invention is used to denote a semiconductor nanocrystal. Each QD typically comprises a core and a cap comprised of different materials, although QDs comprising only one type of material are encompassed by the present invention. Generally, however, the fluorescence emission increases when a core/cap structure is used. Regardless of whether a single material or a core/cap structure is used, the entire QD preferably has a diameter ranging from 0.5 nm to 30 nm, and more preferably from 1 nm to 10 nm.

The “core” is a nanoparticle-sized semiconductor. While any core of the II-VI semiconductors (e.g., ZnS, ZnSe, ZuTe, CdS, CdSe, CdTe, HgS, HgSe, HgTe, and mixtures thereof), III-V semiconductors (e.g., GaAs, InGaAs, InP, InAs, and mixtures thereof) or IV (e.g., Ge, Si) semiconductors can be used in the context of the present invention, the core must be such that, upon combination with a cap, a luminescent quantum dot results. A II-VI semiconductor is a compound that contains at least one element from Group II and at least one element from Group VI of the periodic table, and so on. Preferably, the core is a IIB-VIB semiconductor, a IIIB-VB semiconductor or a IVB-IVB semiconductor that ranges in size from about 1 μm to about 10 nm. The core is more preferably a IIB-VIB semiconductor and ranges in size from about 2 nm to about 5 nm. Most preferably, the core is CdS or CdSe.

The “cap” is a semiconductor that differs from the semiconductor of the core and binds to the core, thereby forming a surface layer or shell on the core. The cap must be such that, upon combination with a given semiconductor core, results in a luminescent quantum dot. Preferably, the cap passivates the core by having a higher band gap than the core, so the excitation of the QD is confined to the core, thereby eliminating nonradiative pathways and preventing photochemical degradation. In this regard, the cap is preferably a JIB-VIB semiconductor of high band gap. More preferably, the cap is ZnS or CdS. Most preferably, the cap is ZnS. In particular, the cap is preferably ZnS when the core is CdSe or CdS and the cap is preferably CdS when the core is CdSe. Other examples of core/cap combinations for QDs include CdS/HgS/CdS, InAs/GaAs, GaAs/AlGaAs and CdSe/ZnS. In general, the cap is 1-10 monolayers thick, more preferably 1-5 monolayers, and most preferably 1-3 monolayers. A fraction of a monolayer is also encompassed under the present invention. For example, a CdS cap 1.3 monolayers thick is especially preferred.

The synthesis of QDs is well known in the art as disclosed, for example, by U.S. Pat. Nos. 5,906,670, 5,888,885, 5,229,320, 5,482,890, and Hines, M. A. J. Phys. Chem., 100, 468-471 (1996), Dabbousi, B. O. J. Phys. Chem. B, 101, 9463-9475 (1997), Peng, X., J. Am. Chem. Soc., 119, 7019-7029 (1997), which are incorporated herein by way of reference.

The wavelength emitted by the QDs can be selected according to the physical properties of the QDs, such as the size of the nanocrystal. QDs are known to emit light from about 300 mm to about 1700 nm. The wavelength band of light emitted by the QD is determined by either the size of the core or the size of the core and cap, depending on the materials comprising the core and cap. The emission wavelength band can be tuned by varying the composition and the size of the QD and/or adding one or more caps around the core in the form of concentric shells.

Each color (i.e., wavelength) of the QD can be embedded in the bead at a predetermined intensity, thereby forming a multicolor QD-tagged bead. For each color, the use of 10 intensity levels (0, 1, 2, . . . 9) gives 9 unique codes (10¹-1), because level “0” cannot be differentiated from the background. The number of codes increases exponentially for each intensity and each color used. For example, a three color and 10 intensity scheme yields 999 codes (10³-1), while a six color and 10 intensity scheme has a theoretical coding capacity of about 1 million (10⁶-1). In general, n intensity levels with m colors generate (n^{m}-1) unique codes. However, the actual coding capabilities are likely to be substantially lower because of spectral overlapping, fluorescence intensity variations, and signal-to-noise requirements. In general, it is more advantageous to use more colors rather than more intensity levels, in order to increase the number of usable codes. The number of intensities is preferably from 0 to 20, more preferably 1-10, more preferably 2-8, more preferably 3-7, more preferably 4-6, more preferably 5-6, and most preferably 6. The number of colors is preferably 1-10 (e.g., 2-8), more preferably, 3-7, and most preferably 5-6. The term “multicolor QD”, is meant that the more than one color of luminescent quantum dots are embedded in the bead. Although preferably more than one color of quantum dot is incorporated in the bead, instances wherein one or more colors' intensity is zero, such as a bead with the red:green:blue code of 1:0:0, is also encompassed by the present invention.

In a preferred embodiment, red, green, and blue QDs are embedded in a bead in a precisely controlled ratio. By the term “precisely controlled ratio”, it is meant that the ratio of intensities for each color of QD used is predetermined before incorporation into the bead. Desirable exact ratios can readily be determined by the ordinary skilled artisan. For example, beads can be embedded with multicolor quantum dots of red, green, and blue in a 1:1:1, 2:1:1, or 2:3:5 (red:green:blue), up to as many intensities desired for each color.

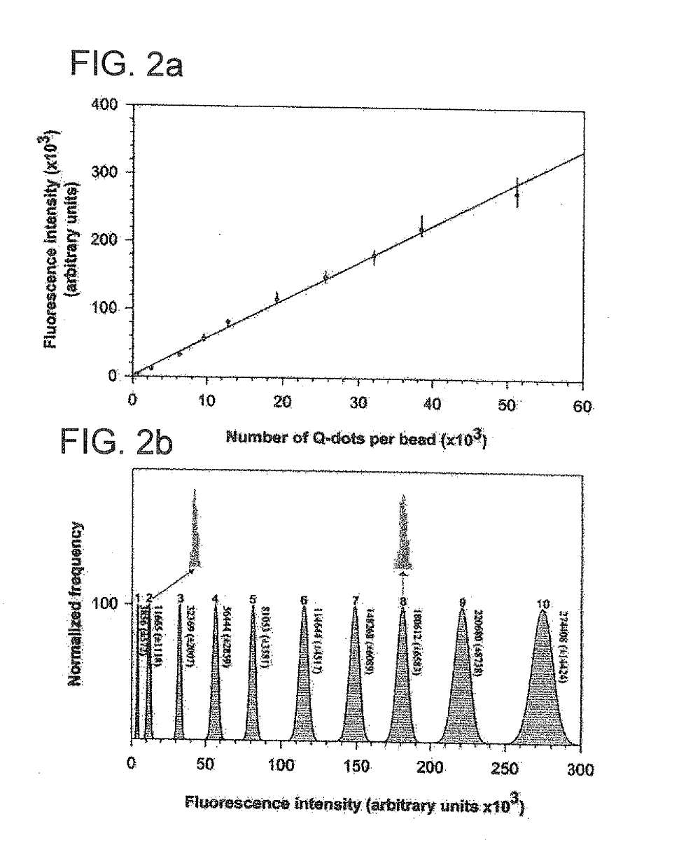

Originally, it was unknown whether or not the embedded QDs would aggregate and couple inside the beads, which could cause spectral broadening, wavelength shifting, and energy transfer. A surprising finding is that the embedded QDs are spatially separated from each other and do not undergo fluorescence resonance energy transfer (FRET). The QDs can either uniformly diffuse throughout the body of beads or penetrate the beads to form fluorescent rings, disks, or other geometrically distinct pattern. The fluorescence spectra of the multicolor QD-tagged beads are narrower by about 10% than that of free QDs, and the emission maxima remain unchanged. Without being bound by any particular theory, it is believed that the bead's porous structure acts as a matrix to spatially separate the embedded QDs, and also as a filter to block the incorporation of large particles in a heterogeneous population. Calculations indicate that the average distance between two adjacent QDs is about 30 nm within a bead having a diameter of 1.2 μm and containing about 50,000 QDs. This calculation suggests that the average separation distance is much larger than the Förster energy transfer radius (Rₒ=5-8 nm) for QDs (Kagan, C. R., et al. Phys. Rev. Lett. 76, 1517-1520 (1996); Micic, O. I., et al. J. Phys. Chem. B, 102, 9791-9796 (1998)). Quantitative and statistical data as shown in FIGS. 2A, B have been obtained on the number of QDs per bead and the fluorescence intensity levels for coding. A linear relationship between the measured fluorescence intensity and the number of embedded QDs (FIG. 2A) further confirms the lack of FRET among the embedded quantum dots, a key requirement for multiplexed optical coding.

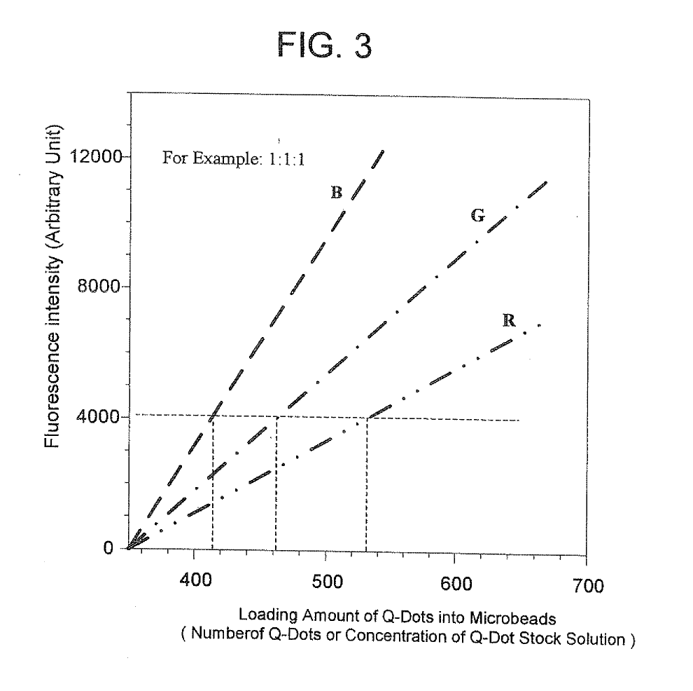

In order to prepare multicolor QD-tagged beads, the uniformity and reproducibility of the tagged beads were analyzed by examining the variations of single-color bead signals and by histogram plots for each of the 10 intensity levels used. As shown in FIG. 2A, the narrow widths in the measured fluorescence intensities indicate a high level of bead uniformity. Statistical analysis of single-bead signals shows that the standard deviations are in the range of 5 to 10%. The histograms in FIG. 2B reveal that there is no intensity overlap among the first six levels at four standard deviations (±4σ), and no overlap among the last four levels at three standard deviations (±3σ). Thus, the bead identification accuracies are estimated to be as high as 99.99% for the first six intensity levels, and about 99.74% for the remaining four levels. These values are statistical accuracies for identifying single-color beads of different intensity levels, not the precision or reproducibility in measuring the absolute fluorescence intensities. Previously, Wild and coworkers have shown that only 500 photons are needed to assign a single fluorescent molecule to one of four species with a confidence level of 99.9% (Prummer, M. et al., Anal. Chem., 72 443-447 (2000)). Working curves for single-color beads such as that in FIG. 2A can be made for each color desired and the curves can be combined (schematic illustration in FIG. 3). Relying on the linear relationship for each color allows for facile determination of how many beads of each color are to be added in order to produce a bead with a desired code.

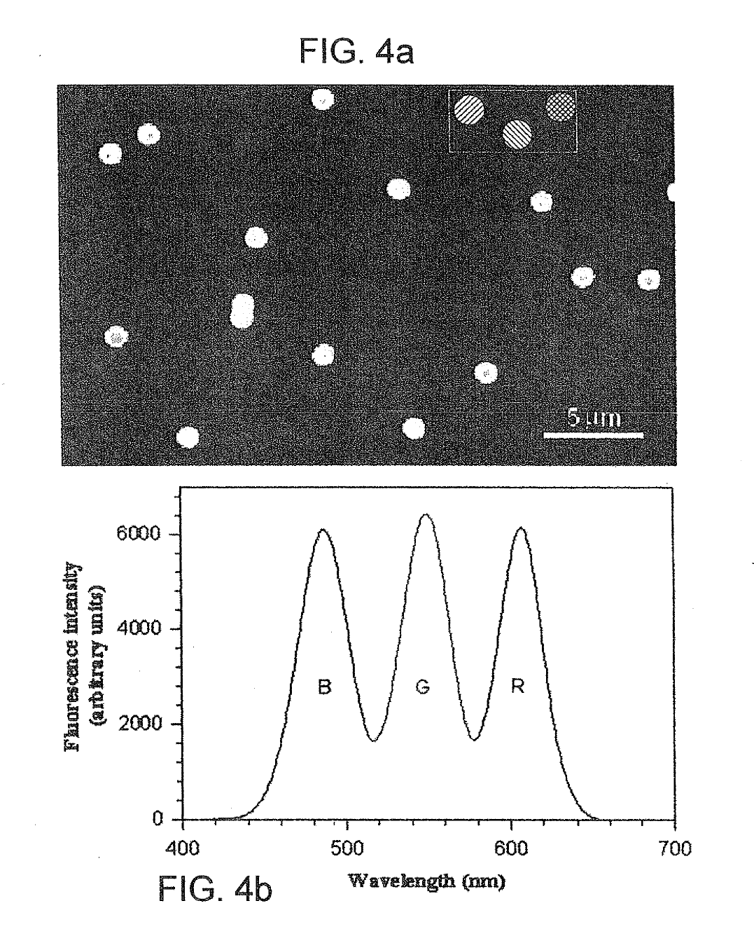

FIG. 4A shows a color image of these triple-color fluorescent beads together with a number of single-color beads. A striking feature is that the triple-color beads appear “white,” because of a precise balance of the emission intensities for all three colors. This balance was achieved by controlling the proportions of different-sized QDs. Single-bead spectroscopy confirmed that the three fluorescence peaks have nearly identical intensities (FIG. 4B). In addition to the amount of QDs in the beads, the color and intensity balances are affected by differences in the optical properties of different-sized QDs, and by the dependence of instrumental response on wavelength. However, all these factors can be compensated by varying the amounts of QDs for each emission color, and this allows empirical rules to be developed for preparing multicolor-tagged beads at predetermined intensity levels. For example, the QD fluorescence spectra are nearly symmetric and can be modeled as a Gaussian distribution. With pre-set emission maxima and intensity levels, spectral deconvolution and signal processing methods should allow code identification under difficult conditions.

In general, the QDs are embedded within the bead, and are only physically held therein by the pore structure of the bead. However, other possible binding modes are possible. For instance, the adherence of the QDs to the bead can occur through covalent, ionic, hydrogen, van der Waals forces and mechanical bonding. Embodiments wherein the QDs are adhered to the surface of the bead (in addition to or instead of embedding within the bead) are encompassed by the present invention.

The QDs embedded in the bead and the target molecule are capable of absorbing energy from, for example, an electromagnetic radiation source (of either broad or narrow bandwidth), and are capable of emitting detectable electromagnetic radiation in a narrow wavelength band when excited. The QDs can emit radiation within a narrow wavelength band of about 40 nm or less, preferably about 20 nm or less, thus permitting the simultaneous use of a plurality of differently colored QDs embedded in the same bead without spectral overlap. Preferably, the QDs are chosen such that their emission spectra do not overlap with the target's emission spectrum.

The embedded QDs must be stable in aqueous conditions and upon exposure to chemical and biochemical reagents. In a preferred embodiment, in order to be stable in an aqueous environment, the porous beads are sealed with a sealant compound. The sealant compound is not particularly limited but should completely seal the bead, not affect the fluorescence of the QDs, and allow for facile direct or indirect attachment of the probe. Silane compounds such as mercaptopropyl-trimethoxysilane, aminopropyltrimethoxysilane, and trimethoxysilylpropylhydrazide are preferred sealant compounds. Unlike free QDs in aqueous buffer, the embedded and protected QDs are stable to the temperature cycling conditions necessary in DNA hybridization assays.

The beads are sealed by any suitable manner. By way of example, and not by way of limitation, the beads are sealed by one of three methods. In the first method, the quantum dot is modified before incorporation into the bead. Both hydrophilic and hydrophobic QDs can be prepared depending on the type of bead (and its interior) used. For example, hydrophilic quantum dots can be coated with silica or mercaptoacetic acid for solubilization. When reacted with CdSe/ZnS nanocrystals in chloroform, the mercapto group binds to a Zn atom, and the polar carboxylic acid group renders the quantum dot water-soluble. Reagents that produce similar results can also be used. Hydrophobic quantum dots can be coated with silane (such as, for example, mercaptopropyltrimethoxysilane, aminopropyltrimethoxysilane, or trimethoxysilylpropylhydrazide), so that the QDs can be dissolved in alcohols or other organic solvents that can suspend microbeads in it such as propanol, butanol, methanol, ethanol, hexanol, dimethylformamide, formamide, and chloroform. Hydrophobic quantum dots capped with TOPO can also be prepared in propanol, butanol, or hexanol, chloroform, or hydrocarbon solvents directly. The modified QDs are embedded in the porous beads. The silane compound on the QI) surfaces is then polymerized inside the bead upon addition of a trace amount of water, thereby sealing the pores. In the second method, the quantum dots are modified after incorporation into the bead with silane (such as, for example, mercaptopropyltrimethoxysilane, aminopropyltrimethoxysilane or trimethoxysilylpropylhydrazide), and then polymerized inside the beads upon addition of a trace amount of water. In the third method, microbeads functionalized with carboxylic or amino groups can be sealed using a silane. For example, aminopropyltrimethoxysilane can be attached to carboxylate (C(O)OH) groups on the bead surface by one step carbodiimide coupling. The silane is then polymerized on the bead surface, thereby completely sealing it. The fourth method is a combination of both the first and third methods or the second and third methods. The QDs are functionalized first, the bead pores are sealed, and then the surface of the bead is sealed.

In the case of using silica microbeads, which are considered non-porous, QDs can be attached on the surface of the microbeads first and then the whole composite can be sealed with a sealant compound (e.g., mercaptopropyltrimethoxysilane, aminopropyltrimethoxysilane, trimethoxysilylpropylhydrazide) and a trace amount of water. If QDs are embedded within the silica beads at the time the bead was synthesized, the bead does not need to be further protected or sealed due to the non-porous nature of the bead. The use of silica beads is less preferred because of their non-porous nature and relatively hydrophilic interiors.

In view of the foregoing, the present invention embodies a multicolor quantum dot-tagged bead, wherein the bead has pores large enough to incorporate quantum dots. The bead can be prepared by emulsion, suspension, or seeded polymerization. Once the QDs are embedded in a predetermined amount, the bead can be sealed with a sealant compound. In a preferred embodiment, the quantum dots are oil-soluble, in other words the QDs are soluble in organic solvents. Preferably, oil-soluble quantum dots are embedded within the interior of a porous bead with pores large enough to incorporate quantum dots, in which the bead has a hydrophobic interior. Because the bead has a hydrophobic interior, the hydrophobic quantum dots will be swept readily into the inside of the bead rather than attach to the bead's surface. The entire portion of the QDs in solution will be embedded within the bead and no quantum dots will remain in solution or on the bead's exterior. This ensures the reproducible production of QD-tagged beads with precisely controlled ratios of embedded QDs.

In another embodiment, the present invention also provides a composition comprising a multicolor quantum dot-tagged bead as described above and a carrier. Any suitable carrier can be used in the composition. Preferably the carrier is aqueous. Desirably, the carrier renders the composition stable at a desired temperature, such as room temperature, and is of an approximately neutral pH. Examples of suitable aqueous carriers are known to those of ordinary skill in the art and include saline solution and phosphate-buffered saline solution (e.g., PBS, TRIS, TBS, MES, BIS-TRIS, ADA, ACES, PIPES, MOPSO, BES, MOPS, TES, HEPES, DIPSO, MOBS, TAPSO, TRIZMA, IIEPPSO, POPSO, TEA, EPPS, TRICINE, GLY-GLY, BICINE, HEPBS, TAPS, AMPD, TABS, AMPSO, CHES, CAPSO, AMP, CAPS, CABS).

In yet another embodiment, the present invention provides a conjugate comprising a multicolor quantum dot-tagged bead prepared as described above and a probe, wherein the probe is attached to the bead. In general several probes of the same type are attached to a single bead. However, multiple probes of different types can be linked to a single bead to allow for the simultaneous detection of multiple targets. In general, 1-50,000 probes are attached to the bead. Preferably 100-30,000 probes are attached, and most preferably 1,000-10,000 probes are attached. The number of probes can be tuned such that the emission from the QDs does not overwhelm the emission of the target (whose emission is directly related to the number of probes). The attachment of the probe to the bead can occur through, for instance, covalent bonding, ionic bonding, hydrogen bonding, van der Waals forces, and mechanical bonding.

The probe is any molecule capable of being linked to the bead either directly or indirectly via a linker. In addition, the probe will have an affinity for the target molecule for which detection is desired. If, for example, the target is a nucleic acid sequence, the probes should be chosen so as to be complementary to a target sequence, such that the hybridization of the target and the probe occurs. The sequences do not need to be entirely complementary; base pair mismatches that interfere with hybridization between the target sequence and the probe sequences are acceptable. However, if the number of mutations is so great that no hybridization can occur even under the least stringent of hybridization conditions, the sequence is not a complementary target sequence. Thus, by the term “substantially complementary,” it is meant that the probes are sufficiently complementary to the target sequences to hybridize under the selected reaction conditions.

Preferably, the probe is a protein (e.g., an antibody including monoclonal or polyclonal), a nucleic acid (both monomeric and oligomeric), a polysaccharide, a sugar, a fatty acid, a steroid, a purine, a pyrimidine, a drug, or a ligand. Lists of suitable probes are available in “Handbook of Fluorescent Probes and Research Chemicals”, (sixth edition), R. P. Haugland, Molecular Probes, Inc., which is incorporated by its entirety herein by way of reference. Particularly preferred probes are proteins and nucleic acids.

Use of the phrase “protein or a fragment thereof” is intended to encompass a protein, a glycoprotein, a polypeptide, a peptide, and the like, whether isolated from nature, of viral, bacterial, plant, or animal (e.g., mammalian, such as human) origin, or synthetic. A preferred protein or fragment thereof for use as a probe in the present inventive conjugate is an antigen, an epitope of an antigen, an antibody, or an antigenically reactive fragment of an antibody. Use of the phrase “nucleic acid” is intended to encompass DNA and RNA, whether isolated from nature, of viral, bacterial, plant or animal (e.g., mammalian, such as human) origin, synthetic, single-stranded, double-stranded, comprising naturally or non-naturally occurring nucleotides, or chemically modified. A preferred nucleic acid is a single-stranded oligonucleotide.

The probe can be attached by any stable physical or chemical association to the bead directly or indirectly by any suitable means. Desirably, the probe is attached to the bead directly or indirectly through one or more covalent bonds. Direct linking of the probe and the bead implies only the functional groups on the bead surface and the probe itself serve as the points of chemical attachment. If the probe is attached to the bead indirectly, the attachment preferably is by means of a “linker.” Use of the term “linker” is intended to encompass any suitable means that can be used to link the probe to the bead containing the multicolor QDs. The linker should not adversely affect the luminescence of the quantum dot or the function of the attached probe. The linker can be either mono- or bifunctional. Preferably, the linker is an amine, carboxylic, hydroxy, or thiol group. Especially preferred linkers also include streptavidin, neutravidin, avidin and biotin. More than one linker can be used to attach a probe. For instance, a first linker can be attached to a bead wherein QDs are embedded. A second linker can be attached to the first linker. A third linker can be attached to the second linker and so on. A probe is generally attached to the terminal linker such that interaction with the environment is possible. In addition, one linker can be attached to the bead (e.g., biotin) and one linker can be attached to the probe (e.g., avidin). In this embodiment, two linkers are joined (e.g., biotin-avidin) to form the conjugate.

If desired, the surface of the bead can be surface-modified by functional organic molecules with reactive groups such as thiols, amines, carboxyls, and hydroxyl. These surface-active reactants include, but are not limited to, aliphatic and aromatic amines, mercaptocarboxylic acid, carboxylic acids, aldehydes, amides, chloromethyl groups, hydrazide, hydroxyl groups, sulfonates, and sulfates.

In accordance with the invention, the linker should not contact the protein probe or a fragment thereof at an amino acid essential to the function, binding affinity, or activity of the attached protein. Cross-linkers, such as intermediate cross-linkers, can be used to attach a probe to the bead containing the QDs. Ethyl-3-(dimethylaminopropyl) carbodiimide (EDAC) is an example of an intermediate cross-linker. Other examples of intermediate cross-linkers for use in the present invention are known in the art (see, for example, Bioconjugate Techniques, Academic Press, New York, (1996)). Attachment of a probe to the bead containing multicolor QDs can also be effected by a bi-functional compound as is known in the art (see, for example, Bioconjugate Techniques (1996), supra).

In those instances where a short linker causes steric hindrance problems or otherwise affect the functioning of the probe, the length of the linker can be increased, e.g., by the addition of from about a 10 to about a 20 atom spacer, using procedures well known in the art (see, for example, Bioconjugate Techniques (1996), supra). One possible linker is activated polyethylene glycol, which is hydrophilic and is widely used in preparing labeled oligonucleotides.

The present invention also provides a method of making a conjugate comprising a multicolor quantum dot-tagged bead and a probe, such as the conjugates described herein. Where the probe is to be directly attached to the bead comprising the multicolor QDs prepared as described above, the method comprises (a) attaching the probe to the bead; and (b) isolating the conjugate. Preferably, the probe is a protein or a fragment thereof or a nucleic acid. In one embodiment of the method, the bead is a cross-linked polymer derived from styrene/divinylbenzene/acrylic acid prepared as described above and the probe is a protein. Alternatively, the method of making the conjugate comprising a multicolor QD tagged bead and a probe comprises the steps of (a) contacting a probe with (i) a linker, all intermediate cross-linker or a bifunctional molecule, and (ii) a multicolor quantum dot-tagged bead; and (b) isolating the conjugate.

Where the probe is to be indirectly attached to the bead containing the multicolor quantum dots prepared as described above, the present invention provides a method comprising (a) attaching a linker to the bead; (b) attaching the probe to the linker; and (c) isolating the conjugate. In one embodiment of the method of indirectly attaching the probe to the bead, the bead is a cross-linked polymer derived from styrene/divinylbenzene/acrylic acid and the linker and probe are proteins. In another embodiment of the method of directly attaching the probe to the bead, the bead is a cross-linked polymer derived from styrene/divinylbenzene/acrylic acid and the linker is streptavidin and the probe is all oligonucleotide. In another embodiment of the method of indirectly attaching the probe to the bead, the linker is a primary amine or streptavidin, the bead is a cross-linked polymer derived from styrene/divinylbenzene/acrylic acid and the probe is a nucleic acid.

Once the probe has been attached to the multicolor quantum dot-tagged bead, the now-formed conjugate is useful in the detection of at least one target molecule. The target molecule is any molecule with an affinity for the probe. In a preferred embodiment, the probe hybridizes to a sufficiently complementary target sequence to determine the presence or absence of the target sequence in a sample. Preferably the target molecule is a biomolecule, such as a protein, nucleic acid, nucleotide, oligonucleotide, antigen, antibody, metal, ligand, portion of a gene, regulatory sequence, genomic DNA, cDNA, and RNA including mRNA and rRNA. The target molecules can be of any length with the understanding that longer sequences are more specific. Preferably the target molecules are of sufficient length or comprise native conformation to hybridize or bind to the probes attached to the multicolor quantum dot-tagged beads.

The target molecules are preferably either directly labeled with a means of detection (e.g., a tag). The tag is any molecule that fluoresces in the visible, ultraviolet, or infrared region and is excited in the same region as the QDs, such as fluorescent dye or biotin (for binding to fluorescently tagged avidin). For example, a useful fluorescent tag is Cascade Blue, which can be simultaneously excited with the embedded QDs at about 350 nm. Other organic dyes include, but are not limited to, Pyrene, Coumarin, BODIPY, Oregon green, and Rhodamine. An all quantum dot system can be synthesized wherein a single QD is used as the analyte signal. In this example, the analyte label does not have to fluoresce blue (as in the case of Cascade Blue); it can be any wavelength as long as it does not overlap with the coding signal. For example, if the coding signal is on the long wavelength side (red side), a blue-emitting QD can be used for the analyte signal. If the coding signal is on the short wavelength side (blue side), a red-emitting QD can then be used as the analyte signal. In another embodiment, the analyte signal can be in the middle of the coding signal if the peaks of coding signal are far apart from each other. The intensity of the signal generated by the tag attached to the target molecule will be in direct proportion to the amount of target present in the sample. It may be necessary to use weak QD coding signals in the multicolor QD-tagged bead in order to detect the target signal at very low concentrations.

Both the coding signal from the multicolor quantum dot-tagged bead and the target analyte are detected by their fluorescence emission. Detection can be performed with any suitable instrument. Preferably, the target is detected using wavelength-resolved spectroscopy combined with a microfluidic channel. In this method, the beads flow through the microfluidic channel in a single-file manner. At each reading only one bead will be detected.

The present invention provides a method of detecting one or more targets in a sample. The method comprises (a) contacting the sample with the present inventive conjugate prepared as described above, wherein the probe of the conjugate specifically binds to a target; and (b) detecting luminescence, wherein the detection of luminescence indicates that the conjugate bound to the target in the sample. By “specifically binds,” it is meant that the probe preferentially binds the target with greater affinity than non-targeted molecules in the sample.

Also provided by the present invention is a method of detecting one or more proteins in a sample. The method comprises (a) contacting the sample with the present inventive conjugate prepared as described above, wherein the probe of the conjugate specifically binds to a protein; and (b) detecting luminescence, wherein the detection of luminescence indicates that the conjugate bound to the protein in the sample.

Preferably, in the method of protein detection, the probe of the conjugate is a protein or a fragment thereof, such as an antibody or an antigenically reactive fragment thereof, and the protein in the sample is an antigen or an epitope thereof that is bound by the antibody or an antigenically reactive fragment thereof. The antigen or epitope thereof preferably is all or part of a virus or a bacterium. Alternatively, the probe of the conjugate is an antigen or an epitope thereof and the protein in the sample is an antibody or an antigenically reactive fragment thereof that binds to the antigen or epitope thereof. The antibody or the antigenically reactive fragment thereof preferably is specific for a virus, a bacterium, or a part of a virus or a bacterium. In yet another alternative embodiment, the probe of the conjugate is a nucleic acid and the protein in the sample is a nucleic acid binding protein, e.g., a DNA binding protein.

Another method provided by the present invention is a method of detecting one or more nucleic acids in a sample. The method comprises (a) contacting the sample with a conjugate prepared as described above, wherein the probe of the conjugate specifically binds to a nucleic acid; and (b) detecting luminescence, wherein the detection of luminescence indicates that the conjugate bound to the nucleic acid in the sample. Preferably, the probe of the conjugate is a nucleic acid. Alternatively, the probe of the conjugate is a protein or a fragment thereof that binds to a nucleic acid, such as a DNA binding protein.

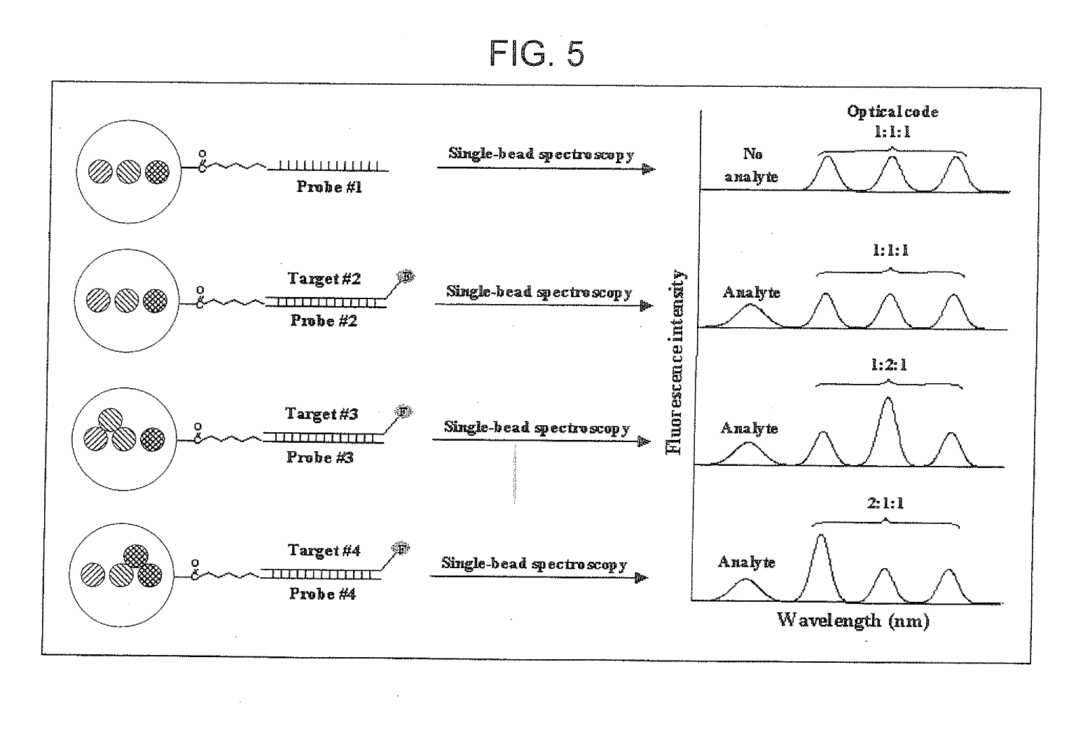

To demonstrate the use of QD-tagged beads for biological assays, a model DNA hybridization system was designed using oligonucleotide probes and triple-color encoded beads, as shown in FIG. 5. Target DNA molecules are either directly labeled with a fluorescent dye or with a biotin (for binding to fluorescently tagged avidin). Optical spectroscopy at the single-bead level (e.g., wavelength-resolved spectroscopy combined with a microfluidic channel) yields both the coding aid the target signals. The coding signals identify the DNA sequence, while the target signal indicates the presence and the abundance of that sequence.

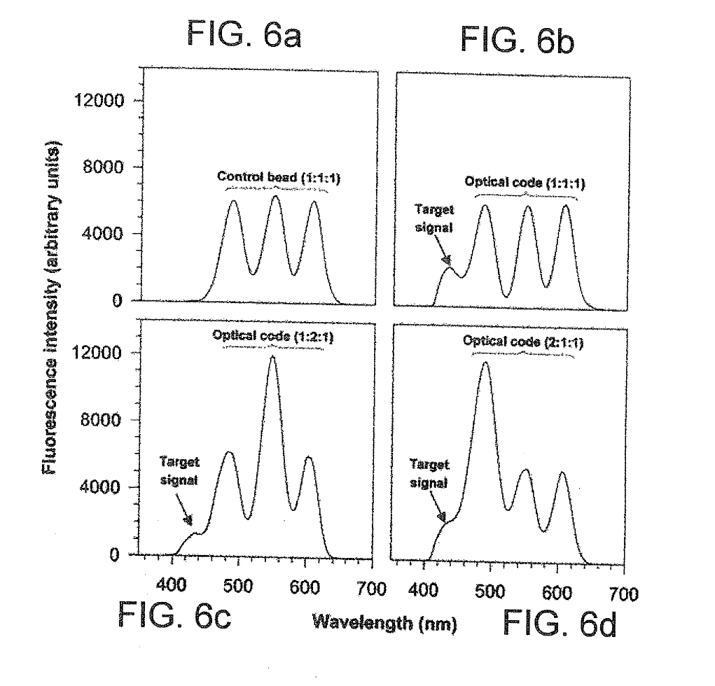

FIG. 6 shows the assay results of one mismatched and three complementary oligos hybridized to triple-color encoded beads. The code 1:1:1 corresponds to the oligo probe 5′-TCA AGG CTC AGT TCC AAT GCA CCA TA-3′. No analyte fluorescence was detected when control oligos (non-complementary sequences) were used for hybridization (A). This result showed a high degree of sequence specificity and a low level of nonspecific adsorption. Analyte fluorescence signals were observed only in the presence of complementary targets, as shown in panels (B) to (D). Assuming 100% efficiencies for both probe conjugation and target hybridization, it was estimated that each bead contained no more than 24,000 probe molecules and no more than 10,000 target molecules.

Preferably, to enhance the accuracy of target detection, the coding and target signals are chosen so their emissions are separated as far as possible to minimize spectral interference caused by overlapping. Under complex biological conditions, the performance (e.g., specificity and sensitivity) of the QD-tagged beads is expected to be similar to that reported by Walt and coworkers. In a recent paper, Walt et al. used 3.1 μm encoded beads to study 25 sequences (including cancer and cystic fibrosis genes) and achieved a detection sensitivity of 10-100 fM target DNA (Ferguson, J. A., et al., Anal. Chem. 72, 5618-5624 (2000)). The underlying principles of nucleic acid hybridization and fluorescence detection are similar, but multicolor QD-tagged bead coding should provide important advantages and applications not available with organic dyes.

Using the present inventive beads, one of ordinary skill in the art will understand that two or more different molecules and/or two or more regions on a given molecule can be simultaneously detected in a sample. The method of detecting two or more different molecules or regions of a single molecule involves using a set of conjugates, wherein each of the conjugates comprises quantum dots of varying colors in different ratios (i.e., codes) attached to a probe that specifically binds to a different molecule or a different region on a given molecule in the sample. Detection of the different target molecules in the sample arises from the unique emission spectrum “code” of the luminescence spectral code generated by the different ratios of quantum dots of which the set of conjugates is comprised. This method also enables different functional domains of a single protein, for example, to be distinguished. Alternatively, a single multicolor tagged bead with different probes attached to it can be used simultaneously to detect two or more different molecules and/or two or more regions on a given molecule.

The method comprises contacting the sample with two or more conjugates, wherein each of the two or more conjugates comprises multicolor quantum dot-tagged beads prepared as described above, and a probe that specifically binds to a different molecule or a different region of a given molecule in the sample. The embedded QDs are in different predetermined ratios and each conjugate has its own unique code based on the ratio of intensities of the multicolor QDs. The method further comprises detecting luminescence, wherein the detection of luminescence of a given spectral code is indicative of a conjugate binding to a molecule in the sample.

In accordance with the present invention, two or more proteins or fragments thereof can be simultaneously detected in a sample. Alternatively, two or more nucleic acids can be simultaneously detected. In this regard, a sample can comprise a mixture of nucleic acids and proteins (or fragments thereof).

Preferably, in the method of detecting two or more proteins or fragments thereof, the probe of each of the conjugates is a protein or a fragment thereof, such as an antibody or an antigenically reactive fragment thereof, and the proteins or fragments thereof in the sample are antigens or epitopes thereof that are bound by the antibody or the antigenically reactive fragment thereof. Alternatively, the probes of each of the conjugates are an antigen or epitope thereof and the proteins or fragments thereof in the sample are antibodies or antigenically reactive fragments thereof that bind to the antigen or epitope thereof. Also preferably, the probe of each of the conjugates is a nucleic acid and the proteins or fragments thereof in the sample are nucleic acid binding proteins, e.g., DNA binding proteins.

Also, in accordance with the present invention, two or more nucleic acids can be simultaneously detected in a sample. Any of the above-described methods for detecting a nucleic acid in a sample can be used with two or more conjugates comprising different ratios of multicolor quantum dots attached to probes that can bind to nucleic acids. Accordingly, one method of simultaneously detecting two or more nucleic acids in a sample comprises (a) contacting the sample with two or more conjugates prepared as described above, in which each conjugate comprises a different ratio of multicolor quantum dots attached to a probe, preferably a nucleic acid, in particular a single-stranded nucleic acid, or a protein or fragment thereof such as a DNA binding protein, that specifically binds to a target nucleic acid in the sample; and (b) detecting luminescence, wherein the detection of luminescence indicates that a conjugate bound to its target nucleic acid in the sample.

In another embodiment of the inventive method of simultaneously detecting two or more molecules in a sample, the sample comprises at least one nucleic acid and at least one protein or fragment thereof. The simultaneous detection of a nucleic acid and a protein or fragment thereof in a sample can be accomplished using the methods described above in accordance with the described methods for detecting a protein or fragment thereof in a sample and the described methods for detecting a nucleic acid in a sample as set forth above.

These methods of detecting multiple targets (or multiple portions of a target) allow for a diagnostic library, wherein the library comprises multiple conjugates prepared as described above that flow through a microchannel or are spread on a substrate surface. The bead of the conjugate may or may not be chemically attached to the substrate surface. The beads can reside on the surface substrate through other non-bonding interactions (e.g., electrostatic interactions). The conjugates comprise probes attached to beads wherein QDs of varying colors are embedded in specific predetermined ratios. The conjugates flow through a microchannel or are spread on a substrate surface by methods known in the art. When the beads are spread on a substrate surface, a map can be created identifying each bead (since each bead has its own unique code) by its fluorescence emission. The library can come in contact with a sample solution containing the target(s). After hybridization, the fluorescence emission spectra will indicate which targets are present in the solution. Once a target is found to be present (or absent) in the sample and its position on the map is identified by the bead code, the identity of the probe will be known. By knowing the identity of the probe, the identity of the target can be found. The diagnostic library can theoretically contain an unlimited number of conjugates. The diagnostic library will comprise at least one conjugate, preferably at least about 100 conjugates, more preferably at least about 500 conjugates, and most preferably at least about 1000 conjugates.

The substrate surface is any suitable material in which the beads comprising the multicolor QDs can be attached. For example, suitable substrates include plastic, glass, ceramic and metal. Examples of plastic substrates include those comprising polyethylene, polystyrene, polytetrafluoroethylene, polycarbonate, polyester, polyether, polyamide, and combinations thereof. Metal substrates include stainless steel, gold, titanium, nickel, and combinations thereof.

In one embodiment of the present invention, a molecular beacon is formed which comprises a conjugate comprising a multicolor quantum dot-tagged bead, a probe, a fluorophore, and a quenching moiety. The probe is a single-stranded oligonucleotide comprising a stem and loop structure wherein a hydrophilic attachment group is attached to one end of the single-stranded oligonucleotide and the quenching moiety is attached to the other end of the single-stranded oligonucleotide. The fluorophore can be any fluorescent organic dye or a single quantum dot such that its emission does not overlap with that of the multicolor quantum dot-tagged bead.

The quenching moiety desirably quenches the luminescence of the fluorophore. Any suitable quenching moiety that quenches the luminescence of the fluorophore can be used in the conjugate described above. The quenching moiety is preferably a nonfluorescent organic chromophore or metal particle, which is covalently linked to the 3′ amino group of the oligonucleotide. Preferably, the quenching moiety is 4-[4′-dimethylaminophenylazo]benzoic acid (DABCYL) or gold or silver particles that are typically 1-10 nm in diameter (see, e.g., Dubertret, B., et al., Nature Biotech., 19, 365-370 (2001); Fang, X., et al., J. Am. Chem. Soc., 121, 2921-2922 (1999); Fang, X., et al., Anal Chem., 72, 3280-3285 (2000)). Preferably, the conjugate comprises a primary amine group at the 3′ end and a biotin group at the 5′ end. Preferably, the multicolor quantum dot-tagged bead is first linked with streptavidin and then conjugated to the 5′ biotin group, preferably at a 1:1 molar ratio.

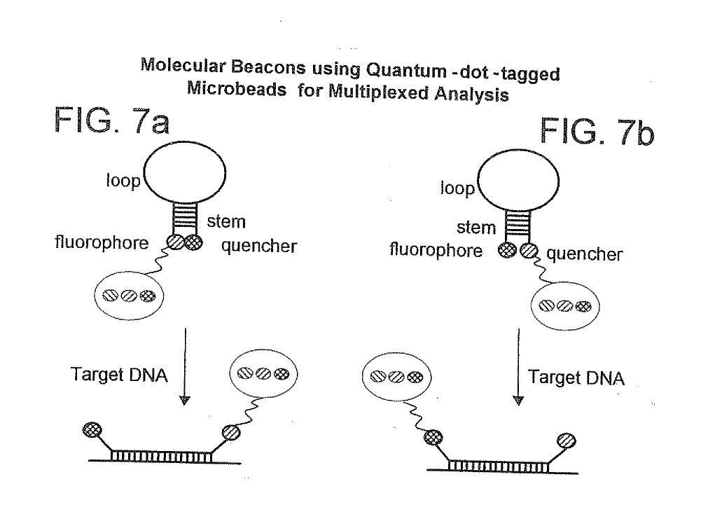

The present invention also provides a method of detecting one or more nucleic acids in a sample using a molecular beacon comprising a single-stranded oligonucleotide having a stem and loop structure, a multicolor quantum dot-tagged bead, a fluorophore, and a quenching moiety. The loop of the oligonucleotide comprises a probe sequence that is complementary to a target sequence in the nucleic acid to be detected in a sample. Desirably, the loop is of sufficient size such that it opens readily upon contact with a target sequence, yet not so large that it is easily sheared. Preferably, the loop is from about 10 nucleotides to about 30 nucleotides, and more preferably from about 15 nucleotides to about 25 nucleotides. The probe sequence can comprise all or less than all of the loop. Preferably, the probe sequence is at least about 15 nucleotides in length. The stem is formed by the annealing of complementary sequences that are at or near the two ends of the single-stranded oligonucleotide. A fluorophore is linked to one end of the single-stranded oligonucleotide and a quenching moiety is covalently linked to the other end of the single-stranded oligonucleotide. A multicolor QD-tagged bead is then attached (either directly or indirectly) to either the fluorophore or the quenching moiety. FIG. 7 illustrates different embodiments of the molecular beacon. The stem keeps the fluorophore and quenching moieties in close proximity to each other so that the luminescence of the fluorophore is quenched when the single-stranded oligonucleotide is not bound to a target sequence. In this regard, the complementary sequences of which the stem is comprised must be sufficiently close to the ends of the oligonucleotide as to effect quenching of the quantum dots. When the probe sequence encounters a target sequence in a nucleic acid to be detected in a sample, it binds, i.e., hybridizes, to the target sequence, thereby forming a probe-target hybrid that is longer and more stable than the stem hybrid. The length and rigidity of the probe-target hybrid prevents the simultaneous formation of the stem hybrid. As a result, the structure undergoes a spontaneous conformational change that forces the stem to open; thereby separating the fluorophore and the quenching moiety and restoring luminescence of the fluorophore. The luminescence of the fluorophore indicates that a target is bound to the probe, and the emission code of the multicolor quantum dot-tagged bead identifies the probe (and hence the target). Using this type of molecular beacon the target itself does not have to be fluorescently labeled, allowing for an even more facile detection of targets.

Accordingly, the method comprises (a) contacting the sample with a conjugate prepared as described above, in which the probe is a single-stranded oligonucleotide comprising a stem-and-loop structure and in which the fluorophore is attached to one end of the single-stranded oligonucleotide, a quenching moiety is attached to the other end of the single-stranded oligonucleotide, and a multicolor quantum dot-tagged bead is attached to either the fluorophore or the quenching moiety, and wherein the quenching moiety quenches the luminescence of the fluorophore, all as described above. The loop comprises a probe sequence that binds to a target sequence in the nucleic acid in the sample. Upon binding, the conjugate undergoes a conformational change that forces the stem to open, thereby separating the fluorophore and the quenching moiety. The method further comprises (b) detecting luminescence of both the fluorophore and the multicolor quantum dot-tagged bead. The detection of the fluorophore luminescence indicates that the conjugate is bound to the nucleic acid in the sample.

Another method includes a method of simultaneously detecting two or more nucleic acids in a sample involves using two or more molecular beacons, each of which comprises a different above-described single-stranded oligonucleotide having a stem-and-loop structure, in accordance with the methods for using such a conjugate as set forth above. The present invention has application in various diagnostic assays, including, but not limited to, the detection of viral infection, cancer, cardiac disease, liver disease, genetic diseases, and immunological diseases. The present invention can be used in a diagnostic assay to detect certain disease targets, by, for example, (a) removing a sample to be tested from a patient; (b) contacting the sample with a multicolor quantum dot-tagged bead conjugate prepared as described above, (c) detecting the luminescence, wherein the detection of luminescence indicates that the disease target is present in the sample. The probe is typically an antibody or antigenically reactive fragment thereof that binds to the virus (e.g., HIV, hepatitis) or protein associated with a given disease state (e.g., cancer, cardiac disease, liver disease). For example, an antibody to HIV gp120 can be used to detect the presence of HIV in a sample; alternatively, HIV gp120 can be used to detect the presence of antibodies to HIV in a sample. The patient sample can be a bodily fluid, (e.g., saliva, tears, blood, serum, urine), cell, or tissue biopsy.

### EXAMPLES

The present invention is described farther in the following examples. These examples serve to illustrate further the present invention and are not intended to limit the scope of the invention.

### Example 1

This example illustrates the formation of polymer beads formed by standard emulsion polymerization.

Polystyrene beads were synthesized by using standard oil and water (o/w) emulsion polymerization at 70° C. in the following methods:

In the first method, the oil phase consisted of styrene (98% v/v), divinylbenzene (1% v/v), and acrylic acid or a derivative such as mono-2-methacryloyloxyethyl succinate (1% v/v) in the presence of the radical initiator AIBN and stabilizer SDS.

In the second method, the oil phase consisted of styrene (93% v/v), divinylbenzene (1% v/v), acrylic acid or a derivative such as mono-2-methacryloyloxyethyl succinate (1% v/v), and 5% dodecane (or octane, decane) in the presence of the radical initiator AIBN and stabilizer SDS. P. A. Lovell, Mohamed S. El-Aasser, “Emulsion polymerization and emulsion polymerization”, Wiley, Inc., (1997).

### Example 2

This example illustrates the formation of porous polymer beads by successive seeded emulsion polymerization.

In this procedure, small latex particles (100200 nm diameter) were grown to larger sizes in the presence of a monomer, an initiator, and an emulsifier. In one example, a mixture was formulated from 10 ml polystyrene seed particles, 20 ml distilled water, 3 ml cyclohexane, 50 μl acrylic acid, 4 ml styrene, 200 μl divinylbenzene, 10 mg benzoyl peroxide, and 30 mg sodium dodecylsulfonate (SDS). The mixture was stirred at room temperature for 18 hours to allow the monomer and the cross-linking reagent to swell the seeds. A stream of nitrogen gas was then purged into the mixture for five minutes, and the temperature of the reaction mixture was raised to 75° C. After 15 hours, the mixture yielded a suspension of polystyrene particles (1-10 μm), with a size distribution of 2-3%.

### Example 3

This example illustrates the formation of porous polymer beads by two-stage seeded polymerization.

In the first stage, 0.2 ml of dibutyl phthalate (DBP) was emulsified within 15 ml of an aqueous medium containing 0.25% (w/w) sodium dodecyl sulfate (SDS). About 1 ml of the aqueous suspension including 120 mg polystyrene seed particles (100-200 nm diameter) was added into the aqueous DBP emulsion. The resulting suspension was stirred at room temperature until all of the emulsified liquid was transferred into the particles (about 5 hours).

In the second stage, DBP-swollen seed particles were further swelled in the monomer phase (containing 0.3 ml of styrene, 0.3 ml of DVB, 10 μl acrylic acid, and 40 mg of benzoyl peroxide). About 0.6 ml of the monomer phase was emulsified by ultrasonication in 15 ml of the aqueous medium. The monomer emulsion was then mixed with the aqueous suspension of DBP-swollen seed particles. The absorption of monomer phase by the DBP-swollen seed particles was stirred at room temperature for 24 h. The resulting emulsion was mixed with 3 ml of a 10% aqueous PVA (polyvinyl alcohol) solution, and purged with bubbling nitrogen for about 5 min. Repolymerization of the monomer phase within the seed particles was carried out on a shaker at 70° C. for 24 h. This two-step procedure yielded uniform and macroporous latex particles in the size range of 1-10 μm (diameter).

### Example 4

This example illustrates the formation of porous polymer beads by suspension (also known as precipitation) polymerization.

Uniform beads were prepared by suspension polymerization in different media and at different initiator concentrations. An ethanol/water or an ethanol/methoxyethanol mixture was used as the suspension medium. In a typical preparation, the suspension medium was obtained by dissolving a proper amount of stabilizer in a mixture of ethanol/water or ethanol/methoxyethanol. The monomer phase was prepared by dissolving the desired amount of initiator within the styrene. The monomer phase was mixed with the suspension medium in a polymerization reactor. The resulting homogeneous solution was purged with bubbling nitrogen for 5 min at room temperature. The polymerization was performed on a shaking water bath at 70° C. for 20 h.

In one example, a dispersed phase was formulated by mixing 0.14 g AIBN, 10 ml styrene, 100 μl acrylic acid, 100 μl divinylbenzene, 10 ml deionized water, 90 ml ethanol, and 1 g PVP (polyvinyl pyrrolidone, MW=40,000). This reaction mixture was degassed with nitrogen for 5 minutes at room temperature before polymerization. When the polymerization was completed, the particles were washed with distilled water to remove the unreacted monomer and other components of the suspension medium.

### Example 5

This example illustrates the incorporation of single-color quantum dots.

The beads were swollen in a solvent mixture containing 5% (v/v) chloroform and 95% (v/v) propanol or butanol and by adding a controlled amount of ZnS-capped CdSe QDs to the mixture. For single-color (such as green) coding with ten intensity levels, the ratios of QDs to beads were in the range of about 640 to about 50,000. The embedding process was complete within about 30 min at room temperature. Alternatively, incorporation of single-color quantum dots was achieved by simply mixing the beads and quantum dots in a solvent mixture containing 5% (v/v) chloroform and 95% (v/v) butanol. Yet another method involved soaking and ultrasonicating porous polymer beads and quantum dots in an alcohol solution, such as butanol or propanol.

### Example 6

This example illustrates the preparation of encoded microbeads with 10-intensities levels.

A working-curve was prepared to determine the relationship between single-bead fluorescence intensities and the number of embedded QDs (see FIGS. 2A, B). Based on this curve, intensity-encoded beads were prepared by using predetermined amounts of QDs in a stock solution. Ten intensity or loading levels were readily achieved by increasing the volume of the QD stock solution proportionally.

### Example 7

This example illustrates the preparation of multicolor encoded beads by sequential QD incorporation.

Incorporation of multicolor-color quantum dots was achieved by swelling the beads in a solvent mixture containing 5% (v/v) chloroform and 95% (v/v) propanol or butanol, and by adding a predetermined amount of multicolor ZnS-capped CdSe QDs to the mixture. For multicolor coding, the amounts of QDs for each color were adjusted experimentally to compensate for the different optical properties of different colored dots. The embedding process was complete within about 30 min at room temperature.

### Example 8

This example illustrates the preparation of multicolor encoded beads by parallel QD incorporation.

Quantum dots of two or more colors were dissolved in an organic solvent mixture at a specifically predetermined ratio. As the beads were swollen in this mixture solvent, multicolor quantum dots were incorporated into the swollen beads simultaneously. As in the case of single-color/ten intensity encoding, working curves for each color could be developed to prepare multicolor-encoded beads at predetermined intensity levels. FIG. 3 illustrates a schematic of how a working curve for each color can be determined. Because of the linear relationship, stock solutions of each desired color can be formulated and added in appropriate amounts to beads to produce the desired ratio.

### Example 9

This example illustrates the protection of the incorporated QDs.

To preserve the optical properties of the embedded QDs under a broad range of experimental conditions, the porous beads were sealed with a thin layer of polysilane, according to a procedure used in bonded-phase chromatography (Dorsey, J. G., et al., Anal. Chem. 66, 857A-867A (1994)). In one embodiment, the encoded beads were protected by using 3-mercaptopropyl trimethoxysilane, which polymerized inside the pores upon addition of a trace amount of water. The quantum dots could be attached to 3-mercaptopropyl trimethoxysilane either before or after incorporation into beads. In another embodiment, the bead surface was protected d by coupling aminopropyltrimethoxysilane to functional carboxylate (or amino) groups by using a carbodiimide cross-linking agent.

### Example 10

This example illustrates the protection of silica beads with QDs attached to the beads' surfaces.

For silica microbeads, quantum dots were first attached to the surface, and were then protected by using mercaptopropyltrimethoxysilane, aminopropyltrimethoxysilane, or trimethoxysilylpropylhydrazide, which polymerized upon the addition of trace water. If QDs are embedded within the silica beads at the time the bead was synthesized, the bead does not need to be further protected or sealed due to the non-porous nature of the bead.

### Example 11

This example illustrates simultaneous QD incorporation and protection

Porous polystyrene/divinyl benzene/acrylic acid beads were soaked and ultrasonicated in a QD solution containing mercaptopropyl trimethoxysilane and tetramethoxysilane. The beads were rinsed to remove any free quantum dots and silane in the solution and on the bead surface. The silane molecules left in the pores were then polymerized upon addition of a trace amount of water.

### Example 12

This example illustrates conjugation of oligo probes with the multicolor quantum dot-tagged bead.

Standard protocols were used to covalently attach the carboxylic acid groups on the bead surface to streptavidin molecules. Nonspecific sites on the bead surface were blocked by using bovine serum albumin (BSA) (0.5 mg/ml) in PBS buffer (pH 7.4). Biotinylated oligo probes (26-mer oligonucleotides, 5′-biotin TEG, HPLC purified, TriLink Biotechnol., San Diego, Calif.) were linked to the beads via the attached streptavidin. Five prime (5′)-biotinylated target oligos were first labeled with avidin-Cascade Blue, and were then hybridized to the oligo probes in 0.1% SDS PBS buffer at 40° C. for 30 min. Prior to fluorescence measurement, the beads were cleaned by two rounds of centrifugation. Both sequential and multiplexed assays yielded similar results. Probe oligos were conjugated to the beads by cross-linking, and target oligos were detected with a blue fluorescent dye such as Cascade Blue. After hybridization, nonspecific molecules and excess reagents were removed by washing.

### Example 13

This example illustrates the detection of a biomolecular target using multicolor quantum dot-tagged beads.

True-color fluorescence images were obtained with an inverted Olympus microscope (IX-70) and a digital color camera (Nikon DI). Broad-band excitation in the near ultra-violet (330-385 nm) was provided by a 100-W mercury lamp. A longpass dichroic filter (DM 400, Chroma Technologies, Brattleboro, Vt.) was used to reject the scattered light and to pass the Stokes-shifted fluorescence signals. A high-numerical-aperture (NA=1.4, 100×), oil-immersion objective was used, and the total wide-field excitation power was about 5 mW.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Of course, variations of those preferred embodiments will become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of preparing a multicolor quantum dot-tagged bead, which method consists essentially of:
(a) providing at least one porous polymer bead, wherein the porous polymer bead is provided by emulsion polymerization, suspension polymerization, or seeded polymerization, and wherein the pores of the bead are large enough to incorporate quantum dots;
(b) combining predetermined amounts of multicolor quantum dots with at least one bead; and
(c) sealing the bead with a sealant compound.

2. The method of claim 1, wherein the multicolor quantum dots are added sequentially.

3. The method of claim 1, wherein the multicolor quantum dots are added in parallel.

4. The method of claim 1, wherein the sealant compound is selected from the group consisting of mercaptopropyltrimethoxysilane, aminopropyltrimethoxysilane, and trimethoxysilylpropylhydrazide.

5. The method of claim 1, wherein the bead is swelled in a solvent.

6. The method of claim 5, wherein the solvent comprises butanol.

7. The method of claim 1, wherein the bead is a cross-linked polymer.

8. The method of claim 7, wherein the cross-linked polymer comprises polystyrene, divinylbenzene, and acrylic acid.

9. A method of preparing a multicolor quantum dot-tagged bead, which method consists essentially of:
(a) providing at least one porous bead by solvent-system polymerization, wherein the solvent-system polymerization includes a styrene monomer, a functionalizing monomer with a terminal COOH, OH, NH₂, or SH group, and about 0.3-5% by volume of a cross-linking agent, wherein the pores of the bead have an average diameter of at least about 1 nm;
(b) combining predetermined amounts of multicolor quantum dots with at least one bead; and
(c) sealing the multicolor quantum dot-tagged bead with a sealant compound.

10. The method according to claim 9, wherein about 1% by volume of the cross-linking agent is added.

11. The method according to claim 9, wherein the cross-linking agent is selected from the group consisting of divinylbenzene, ethylene glycol dimethacrylate, ethylene glycol diacrylate, trimethylolpropane trimethacrylate, and N,N′ methylene-bis-acrylamide.

12. The method according to claim 9, wherein the solvent-system polymerization is precipitation polymerization.


SEQUENCE LISTING (7 sequences)

SEQ ID NO: 1 - DNA, 26 bp, Artificial
  Synthetic
tcaaggctcagttcgaatgcaccata

SEQ ID NO: 2 - DNA, 26 bp, Artificial
  Synthetic
agacaaggtccctgtcaactcttagt

SEQ ID NO: 3 - DNA, 26 bp, Artificial
  Synthetic
tatggtgcattcgaactgagccttga

SEQ ID NO: 4 - DNA, 26 bp, Artificial
  Synthetic
ccgtacaagcatggaacggcttttac

SEQ ID NO: 5 - DNA, 26 bp, Artificial
  Synthetic
gtaaaagccgttccatgcttgtacgg

SEQ ID NO: 6 - DNA, 26 bp, Artificial
  Synthetic
tactcagtagcgacacatggttcgac

SEQ ID NO: 7 - DNA, 26 bp, Artificial
  Synthetic
gtcgaaccatgtgtcgctactgagta